(19) United States
(12) Patent Application Publication
(10) Pub. No.: US 2024/0157271 A1
(43) Pub. Date: May 16, 2024

(54) PROCESS FOR PREPARING ABSOLUTES

(71) Applicant: INTERNATIONAL FLAVORS & FRAGRANCES INC., NEW YORK, NY (US)

(72) Inventors: VANESSA GONNOT, MOUGINS (FR); BERNARD BLEROT, NICE (FR)

(21) Appl. No.: 18/281,782

(22) PCT Filed: Mar. 14, 2022
(86) PCT No.: PCT/US2022/020109

§ 371 Date: Sep. 13, 2023

(30) Foreign Application Priority Data
Mar. 15, 2021 (FR) 2102548

(51) Int. Cl. B01D 11/02; A23L 27/10; A61K 8/9789; A61Q 13/00; C11B 9/02

(52) U.S. Cl. CPC B01D 11/0288; A23L 27/11; A61K 8/9789; A61Q 13/00; C11B 9/025

(57) ABSTRACT

The present invention relates to a process for the eco-responsible preparation of absolutes that may be used in particular as perfume in the cosmetics and perfumery sectors or as flavour in the food sector. This process is characterized by a step of ethanolic extraction of the plant material at very low temperature.

### TECHNICAL FIELD

The present invention relates to a process for the eco-responsible preparation of absolutes that may be used as perfume ingredient in the cosmetics and perfumery sectors or as flavour ingredient in the food industry.

### TECHNOLOGICAL BACKGROUND

Fragrances and perfumes obtained from plant material are highly sought after in the cosmetics industry and in perfumery. They combine three major types of extracts: essential oils obtained by hydrodistillation or by steam distillation, absolutes and resinoids obtained by extraction with an organic solvent and “CO₂” extracts obtained by extraction with supercritical CO₂.

Absolutes are particularly appreciated in perfumery by virtue of their olfactive profile which is more complex than that of essential oils, combining top notes but also base notes provided by non-volatile molecules that are not extracted by hydrodistillation or steam distillation. The odorous molecules present in absolutes are mainly terpenes and terpenoids, but also esters, alcohols, ketones, aldehydes and benzenoids, phenylpropanoids and phenolic compounds.

Absolutes are typically obtained via a process in which the starting plant material is macerated in a volatile organic solvent for a certain time, at room temperature, and the liquid phase is then separated from the plant material. After removal of the solvent by distillation, a residue in the form of a solid or a paste rich in waxes, pigments and odorous molecules is obtained. This residue, known as the concrete, is then dissolved in hot ethanol and the waxes are removed by cold precipitation. After evaporating the ethanol from the filtrate, the absolute is obtained.

The extraction solvents used in the preparation of absolutes are obtained from petrochemistry and mainly combine alkanes such as hexane, heptane or cyclohexane and certain chlorinated solvents such as dichloromethane.

These solvents derived from the petrochemical industry have numerous drawbacks: they are highly flammable, toxic to humans and the environment and are of non-renewable origin.

Nowadays, consumers wish to use cosmetic products based on ingredients of natural origin obtained via processes that are more environmentally friendly, using bio-based materials and avoiding resorting to any products derived from petrochemistry.

There is at the present time a need for novel processes for preparing plant extracts, in particular absolutes, which are more environmentally friendly.

### SUMMARY OF THE INVENTION

One object of the invention is a process for preparing an absolute intended for use as an ingredient in perfumery, cosmetics or food industry (or agri-food) starting with a plant material, said process comprising a step of ethanolic extraction at a temperature below 0° C., and more particularly below −10° C., of the plant material.

In some embodiments, this process comprises:
a) a step of extracting the plant material in ethanol, preferably bioethanol, at a temperature below −10° C. so as to extract the odorous molecules present in the plant material,
b) a step of separating the plant material and the ethanol so as to recover the ethanol containing odorous molecules,
c) a step of removing at least partially the ethanol,
d) optionally a step of removing waxes from the extract obtained in step c), whereby the absolute is obtained.

In some embodiments, step a) is performed by maceration of the plant material in ethanol at a temperature ranging from −15° C. to −50° C., preferably from −20° C. to −40° C., for at least 5 minutes, preferably for at least 15 to 70 minutes.

In a particular embodiment, said step of ethanolic extraction is performed at a temperature below 0° C. and above −10° C. In such a particular embodiment, the process may comprise:
a) a step of extracting the plant material in ethanol, preferably bioethanol, at a temperature below 0° C. and above −10° C., so as to extract the odorous molecules present in the plant material,
b) a step of separating the plant material and the ethanol so as to recover the ethanol containing odorous molecules,
c) a step of removing at least partially the ethanol,
d) optionally, a step of removing waxes from the extract obtained in step c), whereby the absolute is obtained.

In certain embodiments, the extraction step (in particular, step a)) is performed by maceration of the plant material in ethanol for 15 minutes to 120 minutes.

Step b) is typically performed by decantation, pressing, centrifugation and/or filtration of the plant material, preferably at a temperature below −10° C.

Steps a) and b) may be repeated at least once.

In other embodiments, the concentrated extract obtained in step c) has a concrete concentration of from 5% to 15% by volume in ethanol. In step d), the waxes are removed by cold precipitation.

The plant material may be of any type. It may be chosen from fruit, including berries and pods, nuts, buds, flowers, flowering tips, leaves, stems, branches, wood, bark, zests, roots, rhizomes, pips, seeds, pits, and also combinations and fractions thereof. It may be a freshly collected plant material and/or a plant material which has not been subjected to any heat treatment or drying.

In certain embodiments, for example when ethanolic extraction step is performed at a temperature below −10° C., the plant material may be chosen from buds, flowers, flowering tips, leaves, stems and branches.

In certain embodiments, for example when said ethanolic extraction step is performed at a temperature below −10° C., the plant material may be a fresh material.

In certain embodiments, for example when said ethanolic extraction step is performed at a temperature below 0° C. and above −10° C., the plant material may be chosen from a seed, a pip and a pit.

In certain embodiments, for example when said ethanolic extraction step is performed at a temperature below 0° C. and above −10° C., the plant material may be a dry material.

The plant material may in particular be derived from a species chosen from the genera Lavandula, Jasminum, Rosa, Narcissus, Acacia, Ribes, Iris, Citrus, Polianthes, Pogostemon, Ocimum, Pistacia, Myristica, Tagetes, Ilex, Osmanthus, Mentha, Gardenia, Convallaria, Cinnamonum, Pelargonium, Viola, Dipteryx, Gentiana, Triticum, Cananga, Lilium, Hyacinthus, Dianthus, Vanilla, Quercus, Cupressus, Helichrysum, Salvia, Spartium, Cistus, Evernia and Lolium, preferably from Lavandula, Jasminum, Rosa, Narcissus, Acacia, Ribes, Iris, Citrus, Polianthes, Pogostemon, Pelargonium, Viola, Dipteryx, Gentiana, Triticum, Cananga, Lilium, Hyacinthus, Dianthus, Vanilla, Quercus, Cupressus, Helichrysum, Salvia, Spartium, Cistus, Evernia and Lolium.

In certain embodiments, for example when said ethanolic extraction step is performed at a temperature below −10° C., the plant material may be derived from a plant species chosen from Helichrysum, Salvia, Lavandula, Jasminum, Rosa, Acacia, Ribes and Pelargonium.

In certain embodiments, for example when said ethanolic extraction step is performed at a temperature below 0° C. and above −10° C., the plant material may be derived from the plant species Dipteryx.

Preferably, the plant material is not derived from a plant species belonging to the genus Cannabis.

The absolute obtained by the process according to the invention is intended for use as a fragrance in a cosmetic or perfume composition, or as a flavouring in a food product.

For example, the plant material may be obtained from a jasmine species, a lavender species or a rose species.

An object of the invention is also an absolute which may be obtained or which is obtained by the process according to the invention. In certain embodiments, the absolute according to the invention is characterized in that it comprises at least 10%, preferably at least 25%, of terpenic and terpenoid compounds in its composition, these percentages corresponding to the area percentage of the peaks corresponding to the chemical compounds of interest relative to the total area of the peaks in the chromatogram obtained by GC/FID analysis on an apolar column.

In certain embodiments, the absolute according to the invention is characterized in that it comprises at least 10%, preferably at least 25%, of phenylethyl alcohol, these percentages corresponding to the area percentage of the peaks corresponding to the chemical compounds of interest relative to the total area of the peaks in the chromatogram obtained by GC/FID analysis on an apolar column.

In certain embodiments, the absolute according to the invention is characterized in that it comprises at least 10%, preferably at least 25%, of compounds chosen from benzenoids, phenylpropanoids and phenolic compounds and derivatives thereof, these percentages corresponding to the area percentage of the peaks corresponding to the chemical compounds of interest relative to the total area of the peaks in the chromatogram obtained by GC/FID analysis on an apolar column.

The absolute according to the invention is also characterized in that it is free of chlorinated solvents and of alkane or cycloalkane solvents such as hexane, heptane and cyclohexane. In general, the absolute according to the invention is preferably free of any trace of organic solvent other than ethanol.

In certain embodiments, the absolute according to the invention is an absolute of flowers of jasmine, in particular of Jasminum grandiflorum, comprising at least 0.3% of elenolide isomers and at least 3.0% of hydroxytyrosol.

In certain embodiments, the absolute according to the invention is characterized in that it comprises at least 30% of terpenic compounds and is chosen from an absolute of jasmine (e.g. Jasminum grandiflorum or Jasminum sambac), of lavender (e.g. Lavandula angustifolia), of mimosa (e.g. Acacia dealbata), of clary sage (e.g. Salvia sclarea), of pelargonium (e.g. Pelargonium×hybridum) or of blackcurrant (e.g. Ribes nigrum).

In certain embodiments, the absolute according to the invention is characterized in that it comprises at least 45% of benzenoid or phenylpropanoid compounds and/or phenolic compounds and derivatives thereof (such as coumarins, flavonoids and phenolic acids) and in that it is an absolute of tonka (e.g. Dipteryx odorata).

In certain embodiments, the absolute according to the invention is an absolute of rose flowers.

Preferably, this absolute of rose flowers comprises at least 10%, or even at least 25%, of phenylethyl alcohol, these percentages corresponding to the area percentage of the peaks corresponding to the chemical compounds of interest relative to the total area of the peaks in the chromatogram obtained by GC/FID analysis on an apolar column.

In certain embodiments, the absolute according to the invention is an absolute of immortelle flowers, leaves and stems. Preferably, this absolute of immortelle flowers, leaves and stems comprises at least 10% of terpenic and terpenoids (and preferably at least 10% of compounds chosen from benzenoids, phenylpropanoids and phenolic compounds and derivatives thereof), these percentages corresponding to the area percentage of the peaks corresponding to the chemical compounds of interest relative to the total area of the peaks in the chromatogram obtained by GC/FID analysis on an apolar column.

An object of the invention is also the use of an absolute according to the invention as a fragrance or cosmetic active agent, in particular in a perfume or in a cosmetic composition, or alternatively as a flavour, in particular in a composition for food flavouring or in a food product.

### DETAILED DESCRIPTION OF THE INVENTION

The Applicant is a company specialized in the design of plant extracts intended for perfumery. For many years, the Applicant has striven to develop eco-responsible fragrances, by controlling the circuits for the production of its raw materials and by improving its extraction processes.

The solvent most frequently used in the preparation of absolutes is hexane. However, hexane is a solvent derived from petrochemistry, which is particularly toxic to humans and the environment.

The Applicant thus sought an alternative solution to extraction with hexane, which is eco-responsible and which gives an extraction yield and olfactive properties close to those of the absolutes obtained by extraction with hexane.

After extensive research, the Applicant has designed a process for preparing absolutes in which extraction with hexane at room temperature is replaced with an ethanolic extraction step at very low temperature, namely at a temperature below 0° C., and more particularly below −10° C.

As illustrated in the examples, the process developed by the Applicant makes it possible to obtain absolutes which have little colouring, and an olfactive profile that is particularly appreciated by perfumers and which is at least as good as that obtained by extraction with hexane. The yield for the extraction process developed by the Applicant is at least equivalent to or even greater than that of the reference process. As illustrated by the gas chromatography analyses on an apolar column, the absolutes obtained by ethanolic extraction at very low temperature have a composition of terpenoids and terpenes, and/or of benzenoids, phenylpropanoids, phenolic compounds and derivatives thereof, such as coumarins, flavonoids and phenolic acids, close to that of the absolutes obtained by extraction with hexane while at the same time displaying particular molecular signatures that have never been documented to date, such as in the case of the absolutes of Spanish jasmine (or equivalently “jasmine of Grasse”). For example, the absolute of Spanish jasmine obtained by ethanolic extraction at very low temperature contains three elenolide isomers which are not present in the absolute obtained by extraction with hexane. It should be noted that one of these isomers was recently identified in olive oil (Rigakou, Science of Food and Agriculture, 99(12), 5319-5326); the olive tree and jasmine share these secondary metabolites as a result of their common phytochemical family (Oleaceae).

The process developed by the Applicant makes it possible to prepare absolutes which meet certification specifications such as “Agriculture Biologique [Organic Farming] (AB)”, “USDA ORGANIC”, IGP “Absolue Pays de Grasse” and “COSMOS” owing to the use of raw materials derived from organic farming and of bioethanol.

Finally, the process according to the invention is faster than the conventional process, since it is not necessary to isolate the concrete before the step of removing waxes.

The results obtained by the Applicant are very surprising in view of the general knowledge of a person skilled in the art.

Specifically, the use of ethanol as an extraction solvent in the preparation of odorous plant extracts, intended for their use as fragrance or perfume, would have been entirely counter-intuitive to a person skilled in the art, since it is a polar solvent.

Now, the molecules responsible for the olfactive properties of the absolutes are mainly terpenes and terpenoids, and/or benzenoids, phenylpropanoids, phenolic compounds and derivatives thereof, which are known for their hydrophobic nature. In this regard, Chemat et al. predicted using the COSMO-RS software and then confirmed experimentally that ethanol has properties of hot extraction and solvation of terpenic compounds, in particular of monoterpenes, which are very much inferior to those of hexane (Chemat et al. 2019, Separation Science and Technology, 2020, 55, pages 716-727). Moreover, hot ethanol is less inert than hexane and might be responsible for side reactions with odorous molecules.

Thus, for a person skilled in the art, ethanol would not have been a viable alternative to hexane for the preparation of absolutes intended for perfumery.

Moreover, to the Applicant's knowledge, ethanolic extraction at very low temperature has never been used in the field of perfumery for preparing fragrances.

However, the examples show that the extraction temperature has a very significant effect on the olfactive and colorimetric properties of absolutes: the extracts obtained by ethanolic extraction at a temperature above 0° C., or even above −10° C., have a weak and unpleasant odour associated with strong colouring and are thus unsuitable for use as fragrance or perfume.

Interestingly, it has been found that, for flowers, stems, leaves and flowering tips, an optimum extraction in terms of organoleptic quality was obtained at a temperature below −10° C., in particular around −15° C. and −25° C.

For certain dry plant materials, in particular dry fruit or dry seeds, such as tonka bean, an optimum extraction could be obtained at a temperature below 0° C. and above −10° C., in particular around −5° C.

The Applicant notes that cold extraction was proposed in international patent application WO 2020/028992 for preparing cannabinoid-enriched plant extracts intended for therapeutic uses. However, said application does not suggest that ethanolic extraction at very low temperature can be used to prepare absolutes intended for use as perfume or fragrance for the cosmetic industry or perfumery.

Thus, according to a first aspect, the invention relates to a process for preparing an absolute by ethanolic extraction at very low temperature, namely at a temperature below 0° C., and more particularly below −5° C. or −10° C., of a plant material.

More precisely, an object of the invention is a process for preparing an absolute intended for use as an ingredient in a cosmetic, perfume or food composition starting with a plant material, said process comprising:
a) a step of extracting the plant material in ethanol, at a temperature below 0° C. and more particularly below −10° C., so as to extract the odorous molecules present in the plant material,
b) a step of separating the plant material from the mixture of step a) so as to recover the ethanol containing odorous molecules,
c) a step of removing at least partially the ethanol, and
d) optionally, a step of removing the waxes from the extract obtained in step c), whereby the absolute is obtained.

For instance, said process may comprise:
a) a step of extracting the plant material in ethanol, at a temperature below 0° C. and more particularly below −10° C., so as to extract the odorous molecules present in the plant material,
b) a step of separating the plant material from the mixture of step a) so as to recover the ethanol containing odorous molecules,
c) a step of removing partially the ethanol so as to obtain a concentrated extract, and
d) a step of obtaining the absolute from the concentrated extract by removal of the waxes.

In a more particular embodiment, the process may comprise:
a) a step of extracting the plant material in ethanol, preferably bioethanol, at a temperature below −10° C. (e.g. at a temperature between −30° C. and −15° C.) so as to extract the odorous molecules present in the plant material,
b) a step of separating the plant material and the ethanol so as to recover the ethanol containing odorous molecules,
c) a step of removing at least partially the ethanol so as to obtain a concentrated extract,
d) a step of obtaining the absolute from the concentrated extract by removal of the waxes.

In another more particular embodiment, the process may comprise:
a) a step of extracting the plant material in ethanol, preferably bioethanol, at a temperature below 0° C. and above −10° C., so as to extract the odorous molecules present in the plant material,
b) a step of separating the plant material and the ethanol so as to recover the ethanol containing odorous molecules,
c) a step of removing at least partially the ethanol so as to obtain a concentrated extract,
d) a step of obtaining the absolute from the concentrated extract by removal of the waxes.

As a result of the absence of use of toxic solvent in its preparation, the absolutes according to the invention find applications in the cosmetics and perfumery sectors, but also in the food (or agrifood) sector.

The absolutes according to the invention are thus intended for use as ingredient in a food composition, in a cosmetic composition or a perfume.

As a result of their very good olfactive profile, the absolutes obtained by the process according to the invention are typically used as fragrance in a cosmetic composition or a perfume composition or alternatively in aromatherapy. The absolutes obtained by the process according to the invention may also be used as flavour, in particular in a food product.

As explained hereinbelow, the process according to the invention makes it possible to extract classes of odorous molecules which also have the potential to exert biological activity on the skin, in particular a cosmetic effect. Thus, in certain cases, the absolutes obtained according to the process of the invention may be used as active agent with a cosmetic effect. In particular, they may be used both as perfume and as active ingredient exerting a cosmetic effect in a cosmetic composition. The term “cosmetic effect” means any non-therapeutic effect aiming at modifying and/or improving the appearance of the skin or mucous membranes such as the lips, or alternatively preventing and/or correcting non-pathological impairments of the skin or of its integuments.

The term “plant material” means any type of plant material originating from any type of plant species. The term “plant material” depends on the plant species under consideration and covers fruit, including berries, nuts, buds, flowers, flowering tips, leaves, stems, branches, wood, bark, zests, roots, rhizomes, pips, seeds and pits and also combinations and fractions thereof.

In certain embodiments, the plant material comprises or consists of flowers, stems, leaves, buds, flowering tips and combinations thereof.

The “plant material” used in the process according to the invention may be a fresh material, i.e. freshly harvested material, or dry, milled, including cryomilled, freeze-dried or frozen material. In a particular embodiment, the plant material is a fresh material (including a frozen fresh material).

In another particular embodiment, the plant material is a dry material.

The term “dry” or “dried” material refers to plant material that is naturally low in water or which has been subjected to a treatment resulting in a reduction in its water content, such as a drying step.

In a particular embodiment, the plant material is chosen from flowers, flowering tips, berries and pods, nuts, buds, leaves, stems, branches, zests, roots and rhizomes, preferably from buds, flowers, flowering tips, leaves, stems and branches. In such a particular embodiment, the material may be dry or fresh (including frozen fresh material). In certain embodiments, said plant material, preferably buds, flowers, flowering tips, leaves, stems and branches, is fresh.

In another particular embodiment, the material is chosen from wood, bark, pips, seeds and pits, preferably from a seed, a pip and a pit. In such a particular embodiment, the material may be dry or fresh, preferably dry.

The plant material may be subjected to one or more treatments chosen from a washing, chopping or milling, for example cryomilling, or drying step and combinations thereof, before performing the process according to the invention.

In certain embodiments, the plant material is dried and/or is exposed to a heat treatment, in particular to heating, before the step of extraction with ethanol at very low temperature.

In other embodiments, the plant material is washed and optionally milled before performing the process according to the invention. In particular, the plant material is not subjected to any heat treatment and/or any drying treatment.

In a particular embodiment, the plant material used in the process is a fresh material, i.e. freshly harvested material, e.g. material harvested less than 6 hours previously. It may be subjected to a washing and/or chopping or milling step before performing the process. In certain embodiments, the plant material is a fresh material which has at most been subjected to a step of washing in water before performing the process according to the invention.

For example, said material may be leaves, stems, buds, flowers, flowering tips or a combination thereof, which have been freshly harvested.

As indicated above, the plant material may originate from any species. It is preferably a plant of interest for perfumery or the cosmetics industry. Preferably, it is a plant whose extracts are commonly used as perfume or fragrance in cosmetic or perfume compositions.

In certain embodiments, the plant material has been grown by organic farming. Preferably, the plant material is certified “AB” (Agriculture Biologique [Organic Farming]) or “USDA Organic”.

Examples of plants of interest for the present invention are cited later in the description.

The term “absolute” refers to any odorous plant extract, i.e. any extract containing odorous molecules and having an olfactive profile that is suitable for use as a perfume or fragrance. An absolute does not contain any waxes and is generally obtained by a process comprising:
i) a step of extracting the plant material with a volatile organic solvent, and
ii) a step of removing the waxes present in the extract from step a) so as to obtain the absolute.

In some embodiments, only very few amounts of waxes (or even, no waxes) are extracted in step i), such that it is not necessary to perform step ii). In such embodiment, step (i) produces an absolute that can be used as a flavour, perfume or fragrance. Thus, step ii) is optional.

In the context of the present invention, the organic solvent used in step i) is ethanol at a temperature below 0° C., and more particularly below −10° C. The ethanolic extraction step is preferably performed at a temperature comprised between −50° C. and −15° C., for example from −40° C. to −20° C. In certain embodiments, the plant material is chosen from branches, leaves, flowers, stems, flowering tips, buds and mixtures thereof, which are preferably fresh (including frozen fresh), and the ethanolic extraction step (i.e. step a)) is performed at a temperature below −10° C., for example at a temperature comprised between −50° C. and −15° C., or between −40° C. and −20° C., or even between −30° C. and −20° C.

When the plant material is a fresh material (for example fresh leaves, stems, flowers or flowering tips, buds, including frozen fresh), the ethanolic extraction step (i.e. step a)) is preferably performed at a temperature below −10° C., for example at a temperature comprised between −50° C. and −15° C., or between −40° C. and −20° C., or even between −30° C. and −20° C., such as a temperature of −25° C.±2° C.

In certain embodiments, the plant material is a seed, a pip or a pit, in particular a tonka bean, and the ethanolic extraction step is performed at a temperature below 0° C. and above −10° C., in particular at about −5° C.

When the plant material is a dry or dried material, preferably chosen from a seed, a pip or a pit, in particular a tonka bean, the ethanolic extraction step is preferably performed at a temperature below 0° C. and above −10° C., in particular at about −5° C.

The term “odorous molecules” refers to volatile molecules which can produce a perceptible odour, alone or in combination, and which are present in the starting plant material. In the context of the invention, the odorous molecules include, without being limited thereto, the terpenes and terpenoids generally present in plant materials. The odorous molecules may also include benzenoid or phenylpropanoid derivatives, phenolic compounds and derivatives thereof, for example coumarins, flavonoids and phenolic acids.

Terpenes correspond to hydrocarbons resulting from the combination of several isoprene units. They are divided into several classes of compounds depending on the number of isoprene units they contain. In particular, terpenes include monoterpenes, sesquiterpenes, diterpenes and triterpenes. Terpenes may be cyclic or acyclic.

Terpenoids are terpene derivatives comprising additional functional groups, in particular comprising an oxygen (for example an ester, alcohol, ketone or ether oxide function) and/or fewer alkyl groups (for example one less methyl). Terpenoids include, without being limited thereto, terpenic alcohols (or monoterpenols), terpenic ketones and aldehydes, terpenic ethers and terpenic esters. Like terpenes, terpenoids are divided into several classes of compounds depending on the number of isoprene units they contain. Terpenoids may be cyclic or acyclic.

As examples of monoterpenes, mention may be made of ocimenes, limonene, α-pinene, β-pinene, camphene and p-cymene.

As examples of monoterpenoids, mention may be made of terpenic alcohols such as linalool, eucalyptol, menthol, nerol, geraniol, citronellol, borneol, terpineols and acetate esters thereof, or alternatively terpenic ketones and aldehydes such as citral, geranial, citronellal, camphor and carvone.

Other examples of terpenes or terpenoids are in particular β-amyrenone, germanicone, α-amyrine, β-amyrine, lupenone, lupeol, moretenone, moretenol, β-caryophyllene, ar-curcumene, β-selinene, selina-3,11-diene, germacrene D, sclareol, sclareol oxide, cis- or trans-rose oxide, menthone, isomenthone, citronellyl formate, geranyl formate, geranyl tyglate, 10-epi-γ-eudesmol, phytol, hexahydroxyfarnesyl acetone and neophytadiene.

The purpose of step a) is to selectively extract at least a portion of the odorous molecules present in the plant material while at the same time minimizing the extraction of the undesired compounds, in particular the waxes and pigments. This step makes it possible to extract the terpenes, and the terpenoids, in particular the monoterpenes, sesquiterpenes and monoterpenoids such as monoterpenols and monoterpenol acetates, and also the monoterpenic ketones and aldehydes.

Step a) can also make it possible to extract, in addition or alternatively, other odorous molecules of interest such as benzenoid or phenylpropanoid derivatives, phenolic compounds and derivatives thereof, for example coumarins, flavonoids and phenolic acids. Examples of compounds of benzenoid, phenylpropanoid and/or phenolic type are in particular coumarin and 3,4-dihydrocoumarin, eugenol, methyl benzoate, benzyl acetate, and phenylethyl alcohol.

A particular compound chosen from said benzenoid or phenylpropanoid derivatives, phenolic compounds and derivatives thereof, and that may be extracted in step a) is phenylethyl alcohol.

As illustrated in the examples, the absolute obtained from Jasminum grandiflorum grown in Grasse comprises benzyl acetate, benzyl benzoate, cis-jasmone and trans-trans-α-farnesene. The absolute obtained from lavender in particular comprises linalyl acetate, linalool, borneol, β-caryophyllene and coumarin.

The ethanolic extraction step a) may be performed by any means known to a person skilled in the art. For example, step a) may be performed by percolation, i.e. by circulating/percolating at a high flow rate ethanol at a temperature below 0° C., more particularly below −10° C., preferably from −50° C. to −15° C., or, for example, at a temperature above −10° C. and below 0° C., through the plant material, for a certain time, for example for at least 5 minutes, e.g. for at least 10, 15, 30, 60 or even 90 minutes.

In a preferred embodiment, step a) is performed by maceration, i.e. by macerating the plant material in ethanol which has been cooled beforehand to a temperature below 0° C., more particularly below −10° C. The temperature of the mixture thus obtained is maintained at a temperature of at most 0° C., more particularly of at most −10° C., preferably of at most −15° C. or even of at most −20° C. throughout the maceration. As illustrated in the examples, a temperature passing above 0° C., or above −10° C., during the maceration step may have a negative impact on the extraction performance and the olfactive quality of the final absolute. In general, the maceration temperature is within a range from −60° C. to −10° C., preferably from about −50° C. to −15° C. and more preferably from about −45° C. to about −15° C. or from about −40° C. to about −20° C., for example from about −30° C. to about −20° C.

As indicated above, in certain embodiments, in particular when the plant material is a dry material, preferably chosen from a seed, a pip or a pit, in particular a tonka bean, the maceration temperature is advantageously above −10° C. and below 0° C., preferably about −5° C.

When the plant material is a fresh material (for example fresh leaves, stems, buds, flowers or flowering tips, including frozen fresh), the maceration temperature is advantageously below −10° C., for example at a temperature comprised between −50° C. and −15° C., or between −40° C. and −20° C., or even between −30° C. and −20° C., such as a temperature of −25° C.±2° C.

In certain embodiments, the plant material is chosen from leaves, stems, buds, flowers or flowering tips, which are preferably fresh (including frozen fresh), and the maceration temperature is below −10° C., for example at a temperature comprised between −50° C. and −15° C., or between −40° C. and −20° C., or even between −30° C. and −20° C., such as a temperature of −25° C.±2° C.

In certain embodiments, the plant material is a seed, a pip or a pit, in particular a tonka bean, and the maceration step is performed at a temperature below 0° C. and above −10° C., in particular at about −5° C.

For the purposes of the invention, a temperature of about “X” ° C. corresponds to a temperature of “X”±2° C.

In a preferred embodiment, the maceration temperature is maintained at a given temperature ±5° C., preferably ±3° C., e.g. ±2° C.

For example, the temperature may vary within a range from −30° C. to −20° C., e.g. from −28° C. to −22° C. or from −27° C. to −23° C. or else from −26° C. to −24° C. during the maceration.

In certain embodiments, the maceration is performed at a temperature of about −25° C.

Interestingly, it was observed that the temperature range for obtaining an absolute that is acceptable in olfactive terms corresponds to the temperature which makes it possible to obtain a yield of concrete equivalent to that which would be obtained by maceration with hexane at room temperature.

The plant material is macerated in cold ethanol at a temperature below −10° C. for at least 5 minutes. The duration of the maceration step may vary according to the plant material to be extracted and the number of macerations to be performed. In general, the maceration time is at least 5 minutes, for example from 5 minutes to 120 minutes or from 5 minutes to 15 minutes, from 15 minutes to 30 minutes, from 30 minutes to 45 minutes, from 45 minutes to 60 minutes, from 60 minutes to 75 minutes, from 75 minutes to 90 minutes, or alternatively from 90 minutes to 120 minutes, or for instance from 15 minutes to 120 minutes. Thus, for example, the maceration time may be 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes or 75 minutes.

Preferably, the maceration time is less than 65 minutes and may be within a range from 30 minutes to 60 minutes or within a range from 5 minutes to 30 minutes.

The volume of ethanol to be used varies as a function of the equipment and of the raw material to be extracted. In the context of a maceration step a), a volume of ethanol that is sufficient to cover the plant material is generally used. The volume of ethanol is generally within a range from 1 to 20 L/kg, preferably from 2 to 10 L/kg of raw material per maceration cycle.

The maceration may be performed with or without stirring. It is preferably performed without stirring.

In certain cases, the plant material may be subjected to an ultrasonication treatment during step a) to facilitate the release of the odorous molecules. However, in general, such a treatment does not need to be performed.

The ethanol used in the process according to the invention is preferably a bioethanol, i.e. a biobased ethanol. Bioethanol may be obtained by enzymatic hydrolysis of starch originating from cereals, in particular from maize, or by fermentation starting with plants containing sucrose. The ethanol has an alcohol titer of at least 80°, which covers a titer of at least 85°, 90° or even of at least 95°. Preferably, the ethanol has an alcohol degree of at least 90°, typically from 90° to 99°, i.e. a volume percentage of at least 90%, typically from 90% to 99% (v/v). The additional solvent present is preferably water. In step b), the plant material which was extracted and the ethanol containing odorous molecules are separated. This separation may be performed by any method known to those skilled in the art, such as pressing, decantation, filtration or centrifugation. Preferably, it is performed by decantation, centrifugation or pressing optionally followed by a filtration step. These steps are preferably performed at a temperature below 0° C., more particularly below −10° C., typically from −50° C. to −15° C., or, for example, between −10° C. and 0° C.

In certain embodiments, step b) is performed at the extraction temperature used in step a) ±5° C., preferably ±2° C.

Steps a) and b) may be repeated, typically 1, 2 or 3 times, so as to optimize the extraction yield. Thus, on conclusion of the first extraction preferably performed by maceration, the plant material may be recovered and re-extracted in ethanol at a temperature below 0° C., and more particularly below −10° C., preferably by maceration. After the extraction, the liquid phase is recovered and combined with the ethanol recovered from the first extraction. The extraction, preferably maceration, time may be shorter in the additional extraction step(s), while nevertheless being at least 5 minutes. The temperature used in the additional extraction step(s) is below 0° C. or −10° C. and is preferably similar to that used in the first extraction step.

In certain embodiments, steps a) and b) are repeated at least once (e.g. 1, 2 or 3 times) under the same conditions:
the maceration temperature in each step a) is below 0° C. and more particularly below −10° C., preferably between −50° C. and −15° C., e.g. between −40° C. and −15° C., or for example between −10° C. and 0° C.,
the maceration time in each step a) is at least 5 minutes, preferably between 20 minutes and 60 minutes,
the separation of the plant material and of the liquid phase is preferably performed by centrifugation or decantation, followed by a step of filtration of the plant material.

Preferably, steps a) and b) are repeated once.

In step c), the ethanol is at least partially removed so as to obtain a concentrated extract.

The ethanol is preferably at least partially removed by concentration, for example by concentration under reduced pressure or under vacuum.

In certain embodiments, the ethanol is almost totally (or even totally) removed so as to obtain an absolute. In such embodiment, very few amounts of waxes (or even, no waxes) are extracted in step a), such that step d) is not implemented.

When the ethanol is “almost totally removed”, it typically means that it remains at most 2% of ethanol in the obtained absolute (or concrete), the percentage being expressed by volume.

In certain embodiments, the ethanol is almost totally (or even totally) removed so as to obtain a concrete. The concrete is typically in a pasty or solid form rich in odorous molecules and waxes. In this case, depending on the technique used to remove the waxes in step d), it may be necessary to add ethanol so as to obtain a concentrated extract in which the waxes are at least partially soluble at room temperature. Typically, ethanol is added so as to obtain a concentrated extract containing about 5% to 15%, typically about 10%, of concrete in ethanol, the percentages being expressed by volume.

In a preferred embodiment, the ethanol is partially removed so as to obtain a concentrated extract in which the waxes are still partially soluble at room temperature. Indeed, from an industrial viewpoint, it is more advantageous to partially remove the ethanol rather than to totally remove it, if the waxes are removed in step d) by precipitation.

Generally, the ethanol is partially removed so as to obtain a concentrated extract with a titer of about 5% to 15%, typically about 10%, of concrete in ethanol (v/v). If need be, the concentration may be adjusted by adding ethanol.

Step d) aims at removing the waxes present in the concentrated extract so as to obtain the absolute.

This step may also make it possible to remove any pigments present in the concentrated extract.

Various strategies may be used to remove the waxes, i.e. to obtain the absolute, on the one hand, and the waxes, on the other hand.

The absolute may be recovered by performing an extraction with supercritical CO₂: the constituent odorous molecules of the absolute are extracted from the waxes by entrainment with supercritical CO₂ and are recovered by adjusting the temperature and the pressure. In this case, it is preferable to perform step d) on a concrete rather than on a concentrated extract in ethanol.

An alternative solution to extraction with supercritical CO₂ consists in removing the waxes by exploiting their difference in hot and cold solubility in an alcohol.

Thus, in step d), the concentrated extract is homogenized, if necessary at hot temperature (for example at a temperature below 60° C.) and then cooled to a temperature typically below 5° C., for example from −20° C. to 5° C., or from −15° C. to 3° C., for example from −10° C. to 0° C. or from 0° C. to 3° C., so as to precipitate the waxes. The waxes may be removed by filtration. The absolute is subsequently obtained from the collected filtrate by removing the ethanol, for example by concentrating under vacuum. In general, cooling to a temperature of from 0° C. to 3° C. is sufficient to make the waxes precipitate.

During step d), a portion of the odorous molecules of interest may be trapped in the precipitated waxes, and thus lost.

In order to improve the yield of absolute, an additional step may be performed, which aims at recovering the odorous molecules trapped in the precipitated waxes. Typically, the precipitated waxes are taken up in ethanol or in an aqueous-alcoholic solution containing at least 80% and preferably at least 90% ethanol, homogenized at hot temperature, precipitated at cold temperature and filtered off. The filtrate obtained is combined with the filtrate obtained previously. This step may be repeated until satisfactory depletion of the waxes is obtained.

The process for preparing an absolute according to the invention may comprise one or more steps additional to those mentioned previously.

They may be steps for treating the starting plant material by washing, milling, including cryomilling, drying, freeze-drying, screening, freezing and/or thawing.

Depending on the starting plant material, the process may also comprise one or more additional purification steps, preferably chosen from a distillation step, for example molecular distillation or fractional distillation, a precipitation step, a filtration step, an extraction step, in particular liquid-liquid extraction with a suitable aqueous solution, solid-liquid extraction on a solid support that is capable of trapping the compounds to be removed, or alternatively using a subcritical or supercritical fluid, for example supercritical CO₂.

The additional purification step(s) may be performed to improve the olfactive profile of the absolute (e.g. by removing certain unpleasant notes), to remove colouring, and/or to remove certain compounds that are suspected of being allergenic or carcinogenic, such as methyl eugenol.

Furthermore, as indicated above, steps a), b) and d) may be repeated if need be, for example to increase the yield.

The process may also comprise a step of formulation of the final absolute, for example by adding one or more cosmetically acceptable excipients, in particular a cosmetically or dietetically acceptable vehicle or support. For example, the absolute according to the invention may be diluted in a suitable organic or plant solvent, for example triethyl citrate, glycerol, propylene glycol or an oleaginous plant oil, encapsulated in a vectorization system, for example liposomes, or alternatively absorbed onto a solid vehicle.

The process according to the invention, in particular steps a)-b), may be performed by adapting the commercially available extraction equipment. By way of example, it is in particular possible to use or to adapt the low-temperature ethanol extractors sold by the company Delta Separations or the company Devex for the extraction of cannabis. By way of example, the Cryo Ethanol eXtraction System extractor from the company Devex is based on extraction by percolation, whereas the CUP Series Ethanol Alcohol Extraction System system from the company Delta Separations is based on the use of an extractor containing a perforated basket equipped with a motorized rotating axis for centrifuging the plant material after the extraction. Reference may in particular be made to U.S. Pat. No. 10,814,338 which describes a cryoethanol extraction system, the content of which is incorporated herein by reference.

For example, when the extraction in step a) is performed by maceration, the process according to the invention may be performed according to a semi-continuous process using an installation comprising (i) a unit for cooling the ethanol feeding (ii) an extractor, preferably containing a heat-insulated perforated basket, (iii) filtration means, and (iv) means for evaporating off the solvent under vacuum. As indicated above, the plant material may be derived from any type of plant species commonly used for manufacturing fragrances in perfumery.

Examples that may be mentioned include vanilla, mate, frankincense including that from trees of the genus Boswellia, vetiver, myrrh, iris, benzoin, cocoa, coffee, cistus, labdanum, fenugreek, hay, sandalwood, ginger, tree mosses, e.g. oak moss, nutmeg, peppers, saffron, tea tree, cardamom, daffodil, sage (such as clary sage), galbanum, ambrette, basil, broom, immortelle, mastic, marigold, osmanthus, mint, styrax, oak wood, tiare, violet, lavender, lavandin, rose, daffodil, blackcurrant, preferably blackcurrant bud, narcissus, mimosa, lily, hyacinth, carnation, tuberose, jasmine, for example Jasmine sambac or grandiflorum, vanilla, ylang-ylang, orange tree, preferably orange tree flowers (or equivalently “orange flowers” or “orange blossom”), e.g. bigarad orange tree flowers, pelargonium and tonka (or “tonka bean”).

In a particular embodiment, the plant material is obtained from sage (such as clary sage), immortelle, lavender, lavandin, rose, mimosa, jasmine, pelargonium, orange tree (preferably orange tree flowers) and blackcurrant.

In a more particular embodiment, the plant material is obtained from sage (such as clary sage), immortelle, lavender, lavandin, rose, mimosa, jasmine, pelargonium, or blackcurrant.

In another particular embodiment, the plant material is obtained from tonka.

It should be noted that the plant material used in the process according to the invention is not obtained from a plant species belonging to the genus Cannabis, such as Cannabis sativa, Cannabis indica and Cannabis ruderalis.

In a particular embodiment, the plant material is obtained from a species chosen from those of the families Lamiaceae, Oleaceae, Rutaceae, Liliaceae, Fabaceae, Agavaceae, Rosaceae, Lauraceae, Cupressaceae, Geraniaceae, Violaceae, Gentianaceae, Poaceae, Annonaceae, Caryophyllaceae, Oleacea, Fagaceae, Orchidaceae, Annonaceae and Grossulariaceae.

In certain embodiments, the plant material is obtained from a species belonging to one of the following genera: Lavandula, Jasminum, Rosa, Narcissus, Acacia, Ribes, Iris, Citrus, Polianthes, Pogostemon, Ocimum, Pistacia, Myristica, Tagetes, Rex, Osmanthus, Mentha, Gardenia, Convallaria, Cinnamonum, Pelargonium, Viola, Dipteryx, Gentiana, Triticum, Cananga, Lilium, Hyacinthus, Dianthus, Vanilla, Quercus, Cupressus, Helichrysum, Salvia, Spartium, Cistus, Evernia or Lolium. In a particular embodiment, the plant material is obtained from a species belonging to one of the following genera: Lavandula, Jasminum, Rosa, Acacia, Citrus, Pelargonium, Dipteryx, Ribes, Helichrysum, Salvia, Spartium, Cistus, Evernia or Lolium. In a more particular embodiment, the plant material is obtained from a species belonging to one of the following genera: Lavandula, Jasminum, Rosa, Acacia, Helichrysum, Pelargonium, Dipteryx, Ribes, Citrus and Salvia. In an even more particular embodiment, the plant material is obtained from a species belonging to one of the following genera: Lavandula, Jasminum, Rosa, Acacia, Helichrysum, Pelargonium, Dipteryx, Ribes, and Salvia.

In other embodiments, the plant material is obtained from a species chosen from Lavandula angustifolia, Narcissus poeticus, Narcissus jonquilla, Lavandula×intermedia, Ribes nigrum, Citrus aurantium, Polianthes tuberosa, Pogostemon cablin, Ocimum basilicum, Pistacia lentiscus, Myristica fragrans, Tagetes patula, Ilex paraguariensis, Osmanthus fragrans, Gardenia tahitensis, Convallaria majalis, Jasminum grandiflorum, Jasminum sambac, Rosa damascena, Rosa centifolia, Cananga odorata, Gentiana lutea, Pelargonium×hybridum, Acacia dealbata, Acacia mearnsii, Acacia farnesiana, Triticum aestivum, Cupressus sempervirens, Cinnamomum zeylanicum, Iris pallida, Iris germanica, Lilium hybridum, Hyacinthus orientalis, Evernia prunastri, Evernia furfuracea, Mentha piperata, Cistus ladaniferus, Helichrysum italicum, Salvia sclarea, Lolium perenne, Dianthus caryophyllus, Vanilla planifolia, Quercus robur, Quercus alba and Dipteryx odorata. In a particular embodiment, the plant material is obtained from a species chosen from Acacia dealbata, Rosa centifolia, Pelargonium×hybridum, Salvia sclarea, Helichrysum italicum, Dipteryx odorata, Citrus aurantium or Ribes nigrum. In a more particular embodiment, the plant material is obtained from a species chosen from Acacia dealbata, Rosa centifolia, Pelargonium×hybridum, Salvia sclarea, Helichrysum italicum, Ribes nigrum, or Dipteryx odorata.

By way of example, the starting plant material may be lavender (e.g. Lavandula angustifolia) or lavandin (e.g. Lavandula×intermedia) flowers and stems, blackcurrant (e.g. Ribes nigrum) buds, bigarad orange tree (e.g. Citrus aurantium) flowers, tuberose (e.g. Polianthes tuberosa) flowers, jasmine flowers, in particular of Jasminum grandiflorum or Jasminum sambac, or rose flowers, in particular of Damask rose (e.g. Rosa damascena) and May rose (e.g. Rosa centifolia), ylang-ylang (e.g. Cananga odorata) flowers, iris (e.g. Iris pallida or Iris germanica) rhizomes or alternatively mimosa (e.g. Acacia dealbata) flowers and branches.

By way of illustration, the plant material may be mimosa (for example Acacia dealbata) flowers and branches (which are in particular fresh), rose (for example Rosa centifolia) flowers (which are in particular fresh), pelargonium (for example Pelargonium×hybridum) leaves and stems (which are in particular fresh), clary sage (for example Salvia sclarea) flowers, stems and leaves (which are in particular fresh or semi-dried), immortelle (for example Helichrysum italicum) flowers, stems and leaves (which are in particular fresh or dried), orange tree (for example Citrus aurantium) flowers, or blackcurrant (for example Ribes nigrum) buds.

As an additional illustration, the plant material may be mimosa (for example Acacia dealbata) flowers and branches (which are in particular fresh), rose (for example Rosa centifolia) flowers (which are in particular fresh), pelargonium (for example Pelargonium×hybridum) leaves and stems (which are in particular fresh), clary sage (for example Salvia sclarea) flowers, stems and leaves (which are in particular fresh or semi-dried), blackcurrant (for example Ribes nigrum) buds, or immortelle (for example Helichrysum italicum) flowers, stems and leaves (which are in particular fresh or dried).

As an additional illustration, the plant material may be dry hay, or tonka beans (for example Dipteryx odorata), more particularly tonka beans (for example Dipteryx odorata) which are preferably milled. As an additional illustration, the plant material may in particular be jasmine flowers, for example from Jasminum grandiflorum, or lavender flowers and stems, for example from Lavandula angustifolia.

The absolutes obtained by the process according to the invention have an olfactive profile that is just as good as or even better than that of the absolutes obtained by conventional maceration with hexane. The gas chromatography analyses on an apolar column show that the process according to the invention makes it possible to extract the majority of the odorous molecules present in the absolutes obtained by maceration with hexane. These analyses show that ethanol at a temperature below 0° C., and more particularly below −10° C., is able to extract terpene, terpenoid, benzenoid and phenylpropanoid, that is virtually comparable to that of hexane at room temperature.

However, as shown in Example 3, the chromatographic analyses show that the quantitative distribution of the odorous compounds present in the absolutes according to the invention is not identical to those of the absolutes obtained by extraction with hexane.

Moreover, in certain cases, these analyses show that maceration with ethanol at very low temperature makes it possible to extract additional odorous molecules, which are not present in the absolutes obtained by the conventional processes.

In other words, for a given plant material, the absolute obtained by the process according to the invention has a molecular signature which differs from that of the absolutes described in the prior art.

Thus, an object of the present invention is also an absolute which may be obtained by the process according to the invention.

As indicated above, an absolute according to the invention has a composition rich in odorous molecules, in particular in terpenes and terpenoids, and/or in benzenoids, phenylpropanoids, phenolic compounds and derivatives thereof, such as coumarins, flavonoids and phenolic acids.

Preferably, an absolute according to the invention comprises at least 10%, at least 15%, at least 20% or at least 25%, preferably at least 30%, or even at least 35% of terpene and terpenoid compounds in its composition, these percentages corresponding to the area percentage of the peaks corresponding to the chemical compounds of interest relative to the total area of the peaks in the chromatogram obtained by GC/FID analysis on an apolar column, preferably performed under the conditions described in Example 3.

In certain embodiments, the absolute according to the invention comprises at least 30%, for instance at least 45%, or even at least 50%, 55%, 60%, 65%, 70%, 75% or 80% of terpene and terpenoid compounds. For example, it may be an absolute of jasmine (e.g. Jasminum grandiflorum or Jasminum sambac), of lavender (e.g. Lavandula angustifolia), of mimosa (e.g. Acacia dealbata), of orange tree (e.g. Citrus aurantium), of blackcurrant (e.g. Ribes nigrum), of clary sage (e.g. Salvia sclarea), or of pelargonium (e.g. Pelargonium×hybridum). Better still, it may be an absolute of jasmine (e.g. Jasminum grandiflorum or Jasminum sambac), of lavender (e.g. Lavandula angustifolia), of mimosa (e.g. Acacia dealbata), of clary sage (e.g. Salvia sclarea), of pelargonium (e.g. Pelargonium ×hybridum) or of blackcurrant (e.g. Ribes nigrum).

In addition or as a variant, the absolute may comprise at least 10%, at least 15%, at least 20% or at least 25%, preferably at least 30%, or even at least 35% of benzenoid or phenylpropanoid compounds and/or phenolic compounds and derivatives thereof (such as coumarins, flavonoids and phenolic acids) in its composition, these percentages corresponding to the area percentage of the peaks corresponding to the chemical compounds of interest relative to the total area of the peaks in the chromatogram obtained by GC/FID analysis on an apolar column, preferably performed under the conditions described in Example 3. In certain embodiments, the absolute according to the invention comprises at least 45%, or even at least 50%, 55%, 60%, 65%, 70%, 75% or 80% of benzenoid or phenylpropanoid compounds and/or phenolic compounds and derivatives thereof (such as coumarins, flavonoids and phenolic acids). For example, it may be an absolute of tonka (e.g. Dipteryx odorata). In a particular embodiment, the absolute may comprise at least 10%, at least 15%, at least 20%, at least 25%, at least 30%, at least 35% or even at least 40% of phenylethyl alcohol in its composition, these percentages corresponding to the area percentage of the peaks corresponding to the chemical compounds of interest relative to the total area of the peaks in the chromatogram obtained by GC/FID analysis on an apolar column, preferably performed under the conditions described in Example 3. For example, it may be an absolute of rose (e.g. Rosa centifolia).

In a particular embodiment, the absolute may comprise at least 10% of terpenic and terpenoid compounds in its composition (and optionally at least 5% or at least 10% of compounds chosen from benzenoids, phenylpropanoids and phenolic compounds and derivatives thereof), these percentages corresponding to the area percentage of the peaks corresponding to the chemical compounds of interest relative to the total area of the peaks in the chromatogram obtained by GC/FID analysis on an apolar column. For example, it may be an absolute of immortelle (e.g. Helichrysum italicum).

In particular, the absolutes according to the invention are also characterized in that they are free of any trace of solvent other than ethanol, in particular of chlorinated solvents such as dichloromethane, or of alkane solvents such as hexane or cyclohexane.

In certain embodiments, the absolutes according to the invention are certifiable or certified by at least one of the following certifications: “AB” (Agriculture Biologique [Organic Farming]), USDA ORGANIC, COSMOS or IGP “Absolue Pays de Grasse”.

In certain embodiments, an object of the invention is an absolute of jasmine or of lavender which may be obtained by the process according to the invention.

In a particular embodiment, the absolute according to the invention is an absolute of jasmine flowers, preferably of fresh Jasminum grandiflorum flowers. Said absolute may comprise at least 0.3% of elenolide isomers and at least 3.0% of hydroxytyrosol.

Said absolute is in particular characterized in that it comprises 0.3% to 3.0% of elenolide isomers (i.e. of 2H-pyran-5-carboxylic acid, 4-[(1-formyl-1-propen-1-yl]3,4-dihydro-2-oxo-, methyl ester isomers), from 3.0% to 15.0% of hydroxytyrosol and from 30% to 55% of terpenes and terpenoids, according to GC-FID analysis on an apolar column.

The absolute of jasmine according to the invention may more particularly comprise from 0.4% to 2.5% of linalool, from 1.5% to 5.0% of benzyl acetate, from 1.0% to 4.0% of indole, from 0.4% to 2.0% of cis-jasmone, from 0.2% to 1.5% of cis-jasmine lactone and from 0.1% to 1.0% of cis-trans-methyl jasmonate.

In another embodiment, the absolute according to the invention is an absolute of stems and flowers of lavender, preferably fresh stems and flowers of Lavandula angustifolia.

Said absolute is in particular characterized by a terpene content of from 1.0% to 3.5%, a terpenoid content of from 75% to 82% and a content of phenolic compounds of from 1% to 3%. The absolute according to the invention may comprise 40-46% of linalool, and from 23% to 29% of linalyl acetate, according to a GC-FID analysis on an apolar column.

In a particular embodiment, the absolute according to the invention is an absolute of branches and flowers (which are in particular fresh) of mimosa, preferably branches and flowers (which are in particular fresh) of Acacia dealbata. Said absolute may comprise from 6% to 18% of 8-Z-heptadecene, 0.5% to 2.5% of heptadecane, 3% to 6% of nonadecane, 5% to 15% of germanicone, 14% to 40% of lupenone, 5.5% to 8.5% of lupeol co-eluted with α-amyrine.

In a particular embodiment, the absolute according to the invention is an absolute of flowers (which are in particular fresh) of rose, preferably flowers (which are in particular fresh) of Rosa centifolia. Said absolute may comprise 14% to 36% of phenylethyl alcohol, 2.5% to 6% of citronellol, 4% to 6% of geraniol, 4% to 9% of nonadecane and 0.2% to 0.6% of eugenol.

In a particular embodiment, the absolute according to the invention is an absolute of flowers, stems and leaves (which are in particular fresh or dry) of immortelle, preferably flowers, stems and leaves (which are in particular fresh or dry) of Helichrysum italicum. Said absolute may comprise 7.0% to 15.0% of 1-(2,4,6-trihydroxy-3-(3-methylbut-2-en-1-yl)phenyl)ethanone, 0.4% to 1.5% of γ-curcumene, 0.5% to 1.2% of alpha-curcumene, 0.10% to 0.45% of neryl acetate, from 0.5% to 1.7% of β-caryophyllene, and from 0.3% to 1.8% of D-selinene.

In a particular embodiment, the absolute according to the invention is an absolute of flowers, stems and leaves (which are in particular fresh or semi-dried) of clary sage, preferably flowers, stems and leaves (which are in particular fresh or semi-dried) of Salvia sclarea. Said absolute may comprise 80% to 84% of sclareol, 5% to 6% of linalyl acetate and 1% to 2% of linalool.

In a particular embodiment, the absolute according to the invention is an absolute of leaves and stems (which are in particular fresh) of pelargonium, preferably leaves and stems (which are in particular fresh) of Pelargonium×hybridum. Said absolute may comprise 15% to 30% of citronellol, 8% to 20% of geraniol, 0.6% to 2% of cis-rose oxide, 0.3% to 2.0% of menthone, and 1.4% to 6% of 10-epi-γ-eudesmol.

In a particular embodiment, the absolute according to the invention is an absolute of buds (in particular fresh frozen, or frozen then cryomilled) of blackcurrant, preferably of buds (in particular fresh frozen, or frozen then cryomilled) of Ribes nigrum. Said absolute may comprise 0.24% to 0.84% of delta-3-carene, 0.33% to 1.15% of gamma-terpinene, 0.40% to 1.20% of terpinolene, 3.5% to 18.1% of beta-caryophyllene, 1.6% to 8.3% of alpha-humulene.

In a particular embodiment, the absolute according to the invention is an absolute of tonka bean, preferably of Dipteryx odorata. Said absolute may comprise 93% to 98% of coumarin.

The percentages indicated above correspond to the area percentage of the peaks corresponding to the chemical compounds of interest relative to the total area of the peaks in the chromatogram obtained by GC/FID analysis on an apolar column, preferably performed under the conditions described in Example 3.

The absolutes according to the invention may be used as ingredients in various fields such as perfumery, flavourings, agrifood, cosmetics and aromatherapy, in particular since they are free of any trace of chlorinated solvent or of alkane solvent, and can be certified or are certifiable as “Agriculture Biologique [Organic Farming]”. The absolutes according to the invention find applications as fragrance in the preparation of cosmetic products of any type or in the preparation of perfume. The absolutes according to the invention may, in certain cases, be used as active agents with a cosmetic effect. In other embodiments, the absolutes according to the invention may be used as flavour (or “flavouring agent” or “flavour ingredient”) in a food composition (e.g. in cake, biscuit or canned product preparations, or in any other prepared product).

An object of the invention is also a cosmetic composition or a perfume comprising an absolute according to the invention, which are in particular certified COSMOS, “AB” (Agriculture Biologique [Organic Farming]), USDA ORGANIC and/or IGP “Absolue Pays de Grasse”. An object of the invention is also a food product comprising an absolute according to the invention, said food product preferably being certified “AB”.

Other aspects and advantages of the present invention will emerge on reading the examples that follow, which should be considered as illustrative and in no way as limiting.

### EXAMPLES

### Example 1: Preparation of the Absolutes

Preparation of an Absolute of Jasmine According to the Invention (Optimized Process)

650 g of fresh Spanish jasmine (Jasminum grandiflorum) flowers grown in France were placed in a perforated basket and immersed in 4 L of ethanol cooled to the desired temperature (below −10° C.). The flowers were macerated for 30 minutes in the ethanol, without stirring and while maintaining the temperature at the desired value. The flowers were rapidly separated from the solvent by pressing and filtration. The filtrate was recovered. The flowers were made to macerate again in fresh ethanol at the same temperature for 30 minutes. The flowers were rapidly separated from the solvent by pressing and filtration. The recovered filtrate was combined with that from the first maceration. The ethanol was then removed by evaporation under reduced pressure (60° C., 200 mbar) so as to obtain the concrete. The concrete was then dissolved in ethanol so as to reach a concentration of 10% by weight. The solution was then cooled at −18° C. for 12 hours, and filtered on a Buchner funnel with a Celite filter so as to remove the precipitated waxes. The filtrate was recovered. The ethanol was evaporated off at 60° C. under reduced pressure (200 mbar) so as to obtain the absolute.

Total evaporation was performed in step c) so as to be able to evaluate the yield of concrete. This total evaporation can be replaced with partial evaporation on an industrial scale to limit the process costs.

The maceration temperature is within a range from −50° C. to −15° C. A preferred temperature for jasmine is −25° C.

Preparation of an Absolute of Lavender (Lavandula angustifolia, Saralia 22 and 29 Hybrids) According to the Invention, and Study of the Influence of the Maceration Temperature

A process similar to that used for preparing the absolutes of jasmine was performed to prepare absolutes from frozen flowers and stems of lavender grown in France.

Preparation of an Absolute of Mimosa (Acacia dealbata) According to the Invention, and Study of the Influence of the Maceration Temperature

A process similar to that used for preparing the absolutes of jasmine was performed to prepare absolutes from fresh flowers and branches of wild mimosa harvested in France.

Preparation of an Absolute of Rose (Rosa centifolia) According to the Invention, and Study of the Influence of the Maceration Temperature

A process similar to that used for preparing the absolutes of jasmine was performed to prepare absolutes from fresh flowers of rose grown in France.

Preparation of an Absolute of Immortelle (Helichrysum Italicum) According to the Invention, and Study of the Influence of the Maceration Temperature

A process similar to that used for preparing the absolutes of jasmine was performed to prepare absolutes from dry flowers, leaves and stems of immortelle grown in the Balkans.

Preparation of an Absolute of Clary Sage (Salvia sclarea) According to the Invention, and Study of the Influence of the Maceration Temperature

A process similar to that used for preparing the absolutes of jasmine was performed to prepare absolutes from semi-dry flowers, leaves and stems of clary sage grown in France.

Preparation of an Absolute of Pelargonium (Pelargonium ×Hybridum) According to the Invention, and Study of the Influence of the Maceration Temperature

A process similar to that used for preparing the absolutes of jasmine was performed to prepare absolutes from fresh stems and leaves of pelargonium grown in France.

Preparation of an Absolute of Blackcurrant (Ribes nigrum) According to the Invention, and Study of the Influence of the Maceration Temperature

A process similar to that used for preparing the absolutes of jasmine was performed to prepare absolutes from frozen buds of blackcurrant (cryomilled or not) grown in France.

Preparation of an Absolute of Tonka (Dipteryx odorata) According to the Invention, and Study of the Influence of the Maceration Temperature

A process similar to that used for preparing the absolutes of jasmine was performed to prepare absolutes from tonka beans grown in Brazil.

Study of the Impact of the Maceration Temperature

A process similar to that described above was performed, testing various maceration temperatures: −50° C., −35° C., −25° C., −15° C., −10° C., −5° C., 10° C. and/or 25° C. Two or only one maceration step were performed.

Preparation of Absolutes by Extraction with Hexane (Conventional Protocol)

Fresh flowers of Jasminum grandiflorum from Grasse, frozen flowers and stems of Lavandula angustifolia of hybrid varieties Saralia 22 and 29, fresh flowers and branches of mimosa (Acacia dealbata), dry flowers, leaves and stems of immortelle (Helichrysum italicum), semi-dry stems, leaves and flowers of clary sage (Salvia sclarea), fresh stems and leaves of pelargonium (Pelargonium ×hybridum), and fresh flowers of rose (Rosa centifolia) were macerated in hexane (about 10 L per kg of flowers) at room temperature (25° C.) for about 30 minutes. The mixture was decanted and the liquid phase and the plant material were separated. The plant material was macerated again in hexane for 30 minutes. The liquid phases from the two macerations were pooled and the hexane was evaporated off so as to obtain the concrete.

The concrete was then taken up in ethanol. The mixture was homogenized, heated slightly and then cooled so as to precipitate the waxes. The mixture was then filtered. The filtrate was recovered. The waxes were taken up in ethanol. The mixture was homogenized at hot temperature, cooled so as to precipitate the waxes, and filtered.

The collected filtrates were combined and the solvent was evaporated off under reduced pressure so as to obtain the absolute.

Preparation of Absolutes by Extraction with Nappar6/Isopropyl Acetate (Conventional Protocol)

Tonka beans were macerated in a mixture of 80% (by mass) Nappar 6/20% (by mass) isopropyl acetate (about 13.5 L per kg of beans) at 35° C. for 1 hour.

The plant material was macerated once again, in the Nappar 6/isopropyl acetate mixture for 1 hour.

The liquid phases from the two macerations were pooled and the solvent was evaporated off so as to obtain the concrete.

The concrete was then taken up in ethanol. The mixture was homogenized, heated slightly and then cooled so as to precipitate the waxes. The mixture was then filtered. The filtrate was recovered. The waxes were taken up in ethanol. The mixture was homogenized at hot temperature, cooled so as to precipitate the waxes, and filtered.

The collected filtrates were combined and the solvent was evaporated off under reduced pressure so as to obtain the absolute.

Preparation of Absolutes by Extraction with Dichloromethane (Conventional Protocol)

Frozen and cryomilled blackcurrant buds were macerated in dichloromethane (about 10 L per kg of flowers) at room temperature (25° C.) for about 30 min. The mixture was decanted and the liquid phase and plant material were separated. The plant material was macerated once again in dichloromethane for 30 min. The liquid phases of the two macerations were combined and the solvent was evaporated so as to obtain the concrete.

The concrete was then taken up in ethanol. The mixture was homogenized, slightly heated then cooled so as to precipitate the waxes. The mixture was then filtered. The filtrate was collected. The waxes were taken up in ethanol. The mixture was homogenized under hot temperature, and cooled so as to precipitate the waxes and filtered.

The collected filtrates were combined and the solvent evaporated under reduced pressure to obtain the absolute.

### Example 2: Olfactive Evaluation and Colorimetric Appearance of the Absolutes

Olfactive Evaluation: Protocol

The absolutes obtained, diluted beforehand to 5% in alcohol, are evaluated by a panel of perfumers.

The first evaluation is done immediately after soaking a blotter in the dilute solution.

During the evaluation, the absolutes obtained by extraction with ethanol are compared with the “control” absolutes obtained by extraction with hexane (or Nappar6/isopropyl acetate or dichloromethane) so as to assess their hedonic pertinence and their power.

The absolutes are then monitored over time in order to assess their olfactive profile, up to 15 hours after soaking on the blotter.

Absolutes of Jasminum grandiflorum

Absolutes obtained from fresh flowers with a single maceration step, variable maceration temperature and time:

TABLE 1

| | | | | | | Control 1 |
|---|---|---|---|---|---|---|
| Reference | Extract 1 | Extract 2 | Extract 3 | Extract 4 | Extract 5 | (hexane) |
| Extraction | 10 min | 30 min | 30 min | 40 min | 30 min | 30 min |
| time | | | | | | |
| Extraction | −50 | −50 | −20 | −5 | 25 | 25 |
| temperature | | | | | | |
| (° C.) | | | | | | |
| Yield | 0.36% | 0.40% | 0.41% | 2.06% | 3.70% | 0.43% |
| concrete | | | | | | |
| Yield | 0.14% | 0.20% | 0.30% | ND | Too many | 0.15% |
| absolute | | | | | waxes - | |
| | | | | | impossible | |
| | | | | | to obtain the | |
| | | | | | absolute | |
| Olfactive | Close to classic | Close to classic | Closest to hexane | Flat, | Too green, | More fruity |
| Evaluation | jasmine absolute, | jasmine absolute | control, delicate, | animalic, | almost | banana, |
| | slightly too | but a pyrazine/ | less chemical, more | hay, dry, | violet leaf, | impactful, |
| | animalic, spicy, | potato/green note | natural, solar, lacks | less | hay, | more volume, |
| | fruity, lacks a bit | on top, becomes | a bit the fruity notes | defined | animalic, | green |
| | in performance: | better after some | and impact on top, | | algue | |
| | very poor | time, more powerful | but effect gardenia | | | |
| | longlasting | | intense, persistent | | | |
| Olfactive | + | ++ | +++ | −−− | −−− | +++ |
| note | | | | | | |
| Colour at | Very pale | Very pale | Very pale | Dark | Dark | Very pale |
| 5% in | yellow | yellow | yellow | yellow | yellow | yellow |
| alcohol | | | | | | |

Absolutes Obtained from Fresh Flowers with Two 30-Minute Maceration Steps, Variable Temperature:

TABLE 2

| | | | | Control 2 |
|---|---|---|---|---|
| Reference | Extract 6 | Extract 7 | Extract 8 | (hexane) |
| Extraction | −50 | −25 | 10 | 25 |
| temperature (° C.) | | | | |
| Yield concrete | 0.47% | 0.73% | 2.78% | 0.73% |
| Yield absolute | 0.24% | 0.54% | 1.95% | 0.14% |
| Olfactive | Slightly seaweed, | More fruity, more | Metallic, dirty, | The most benzyl |
| Evaluation | less jasmine, less | rich, more powerful, | salty, algue | acetate, slightly |
| | rich, more dry | floral, slightly tea | | naphthaline, more |
| | hay, dirty top note | with the tuberose facet; | | powerful but has |
| | | but a dirty top note | | a fatty note |
| Olfactive note | + | +++ | −−− | ++ |
| Colour at 5% | Pale yellow | Orange-yellow | Dark orange- | Pale yellow |
| in alcohol | | | yellow | |

Absolutes of Lavandula angustifolia, Saralia Variety, Saralia 22 and 29 Hybrids

Absolutes obtained from fresh frozen flowers and stems with two 30-minute maceration steps, variable temperature:

TABLE 3

| | | | | | Control 3 |
|---|---|---|---|---|---|
| Reference | Extract 9 | Extract 10 | Extract 11 | Extract 12 | (hexane) |
| Extraction | −50 | −25 | 10 | 25 | 25 |
| temperature (° C.) | | | | | |
| Yield concrete | 0.25% | 0.31% | 1.30% | 1.82% | 0.40% |
| Yield absolute | 0.0045%* | 0.20% | 0.55% | 0.76% | 0.11% |
| Olfactive | The closest to | Close to hexane | Fatty, buttery | Dry hay, | Good on top but |
| Evaluation | hexane absolute, | absolute but | | dirty, feet | after lacks the |
| | has a coumarin | more camphor, | | | coumarin effect |
| | aspect | fresh lavender, | | | of the classical |
| | | slightly burnt, | | | Lavender |
| | | animalic, hay | | | |
| Olfactive note | +++ | ++ | −− | −−− | +++ |
| Colour at 5% | Orange-yellow | Pale green | Dark green | Dark green | Greenish yellow |
| in alcohol | | | | | |

*Accidental loss during handling

Absolutes of Mimosa (Two Maceration Steps)

TABLE 4

| | | | | | Control 4 |
|---|---|---|---|---|---|
| Reference | Extract 13 | Extract 14 | Extract 15 | Extract 16 | (hexane) |
| Extraction time | 2 × 10 min | 2 × 30 min | 2 × 30 min | 2 × 30 min | 2 × 30 min |
| Extraction | −25 | −25 | −15 | −5/0 | 25 |
| temperature (° C.) | | | | | |
| Yield concrete | 0.18% | 0.23% | 0.43% | 0.39% | 0.93% |
| Yield absolute | 0.12% | 0.20% | 0.26% | 0.31% | 0.28% |
| Olfactive | Cake, resinous, | Floral note, close | Powdery, dry, | Cake, almond, | Powdery, not |
| Evaluation | ciste, sugary, | to the actual flower, | mimosa, | benzaldehyde | as strong, |
| | plum | but lacking the | animalic | off notes | vanilla facet |
| | | strength; it has a | | | |
| | | powdery, almond | | | |
| | | facets, with green | | | |
| | | violet nuances | | | |
| Olfactive note | + | +++ | ++ | − | ++ |

Absolutes of Rose (One Maceration Step, Maceration Time: 30 Minutes)

TABLE 5

| | | | | Control 5 |
|---|---|---|---|---|
| Reference | Extract 17 | Extract 18 | Extract 20 | (hexane) |
| Extraction | 25 | −5 | −25 | 30 |
| temperature (° C.) | | | | |
| Yield concrete | 3.27% | 0.84% | 0.38% | 0.25% |
| Yield absolute | 1.00% | 0.25% | 0.15% | 0.08% |
| Olfactive | Less interesting, | Second best | Best sample, rose | Rich rose profile |
| Evaluation | flat, off notes: | sample after | in the garden, | with sweet honey |
| | boiled, solvent, | extract 20, but | natural, aquatic, | top note, typical |
| | plastic | less powerful | ozonic on the dry. | spiciness and |
| | | | Lacks power and | fruity facets |
| | | | still slightly boiled | |
| Olfactive note | −− | + | ++ | +++ |

Absolutes of Immortelle (One Maceration Step, Maceration Time: 30 Minutes)

TABLE 6

| | | | | | Control 6 |
|---|---|---|---|---|---|
| Reference | Extract 21 | Extract 22 | Extract 23 | Extract 24 | (hexane) |
| Extraction | 25 | −5 | −15 | −25 | 25 |
| temperature (° C.) | | | | | |
| Yield concrete | 2.40% | 1.69% | 1.28% | 1.06% | 1.28% |
| Yield absolute | 2.29% | 1.57% | 1.23% | 1.05% | 0.87% |
| Olfactive | Too fruity on top, | Too fruity | Herbaceous note, | Not enough | Characteristic |
| Evaluation | floral aldehydic, | on top | with floral and fruity | immortelle | of immortelle but |
| | cacao valerianic | | facets, close to the | character | not as good as |
| | | | benchmark, slightly | | catalogue quality |
| | | | less powerful and | | (3 macerations are |
| | | | missing a bit the | | normally necessary) |
| | | | salty and spicy facets | | |
| Olfactive note | −− | − | ++ | + | + |

Absolutes of Clary Sage (One Maceration Step, Maceration Time: 30 Minutes)

TABLE 7

| | | | | | Control 7 |
|---|---|---|---|---|---|
| Reference | Extract 25 | Extract 26 | Extract 27 | Extract 28 | (hexane) |
| Extraction | 25 | −5 | −15 | −25 | 25 |
| temperature (° C.) | | | | | |
| Yield concrete | 6.46% | 3.63% | 3.17% | 3.00% | 3.15% |
| Yield absolute | 5.40% | 2.93% | 3.16% | 2.95% | 2.73% |
| Olfactive | Dirty off- | Apricot, | Weak, | Herbal note, less | Characteristic |
| Evaluation | notes, chlorine, | not good, | apricot | powerful than | of clary sage but |
| | swimming pool, | dirty | | benchmark, less | not as good as the |
| | cassis, cat pee | | | warm and fruity | catalogue quality |
| | | | | and reminiscent | (2 longer macerations |
| | | | | of lavender | are normally necessary) |
| Olfactive note | −− | − | + | ++ | + |

Absolutes of Pelargonium (One Maceration Step, Maceration Time: 30 Minutes)

TABLE 8

| | | | | | Control 8 |
|---|---|---|---|---|---|
| Reference | Extract 29 | Extract 30 | Extract 31 | Extract 32 | (hexane) |
| Extraction | 25 | −5 | −15 | −25 | 25 |
| temperature (° C.) | | | | | |
| Yield concrete | 0.34% | 0.23% | 0.26% | 0.19% | 0.08% |
| Yield absolute | 0.24% | 0.17% | 0.18% | 0.15% | 0.09% |
| Olfactive | Lacks | Lacks a bit | Lacks a bit | Green, woody, | Green note, |
| Evaluation | aromatic | aromatic part, | aromatic part, | metallic | almost metallic, |
| | part, plastic | floral rose, | green, floral rose, | facet, almost | lacks the fruity |
| | note | minty character, | minty character, | resinous | floral aromatic part, |
| | | fruity notes | fruity notes | | flat; different from |
| | | | | | catalogue (longer |
| | | | | | macerations needed) |
| Olfactive note | − | ++ | ++ | + | + |

Absolutes of Tonka (One Maceration Step)

TABLE 9

| | | | | Control 9 | |
|---|---|---|---|---|---|
| | | | | (Nappar 6/ | |
| Reference | Extract 33 | Extract 34 | Extract 35 | isopropyl acetate) | |
| Extraction | 15 min | 30 min | 15 min | 2 × 1 | h |
| duration | | | | | |
| Extraction | −5 | −5 | −15 | 35° | C. |

| | | | | |
|---|---|---|---|---|
| temperature (° C.) | | | | |
| Yield concrete | 1.39% | 4.02% | 1.04% | 20% |
| Yield absolute | 0.90% | 2.78% | 0.58% | 3.80% |
| Olfactive | Weak, a | Coumarine, tonka, | Mushroom, | Warm, gourmand, |
| Evaluation | bit tonka | but with slightly | opoponax, myrrh, | almond character, |
| | | mushroom and | plastic off notes | slightly dry, sweet, |
| | | plastic off notes | | powerful, and long- |
| | | | | lasting honey tobacco |
| | | | | profile with milky |
| | | | | praline undertones. |
| Olfactive note | − | + | − | +++ |

Absolutes of Tonka (Two Maceration Steps, Each Maceration Time: 1 Hour)

TABLE 10

| | | | | Control 9 |
|---|---|---|---|---|
| | | | | (Nappar 6/ |
| Reference | Extract 36 | Extract 37 | Extract 38 | isopropyl acetate) |
| Extraction | 25 | −5 | −25 | 35° C. |
| temperature (° C.) | | | | |
| Yield concrete | 10.24% | 5.98% | 3.79% | 20% |
| Yield absolute | 7.16% | 3.73% | 2.76% | 3.80% |
| Olfactive | Too | Recognizable | Slightly | Warm, gourmand, |
| Evaluation | benzaldehyde, | tonka note, with | coumarin, | almond character, |
| | bitter almond, | sweet, balsamic | but not | slightly dry, sweet, |
| | strong on top | character | enough tonka | powerful, and long- |
| | | and almondy | character | lasting honey tobacco |
| | | facets on top | | profile with milky |
| | | | | praline undertones. |
| Olfactive note | − | ++ | − | +++ |

Absolutes of Blackcurrant Buds

TABLE 11

| | | | | | Control | |
|---|---|---|---|---|---|---|
| Reference | Extract 39 | Extract 40 | Extract 41 | Extract 42 | (dichloromethane) | |
| Starting material | Frozen | Frozen | Frozen | Frozen | Frozen | |
| | whole buds | whole buds | milled buds | milled buds | milled buds | |
| Extraction | −15 | −5 | −5 | −5 | 25° | C. |
| temperature (° C.) | | | | | | |
| Extraction time | 2 × 30 min | 2 × 30 min | 1 × 15 min | 2 × 30 min | 2 × 30 | min |

| | | | | | |
|---|---|---|---|---|---|
| yield concrete | 1.61% | 1.33% | 2.08% | 3.86% | 6.46% |
| yield absolute | 0.80% | 0.84% | 1.40% | 2.39% | 3.01% |
| Olfactive | Most | Exotic fruit, | Less | More fruity, | Has a |
| Evaluation | characteristic | less sulfur | blackcurrant, has | exotic fruits, | blackcurrant |
| | of blackcurrant, | | algue & calone | fresh on top | syrup facet, |
| | slightly ether | | notes, less | | delicious |
| | on top | | powerful, plastic | | |
| Olfactive note | +++ | + | − | + | ++++ |

### Conclusion

The absolutes obtained by maceration in ethanol at a temperature below −10° C. offer an olfactive profile close to that of conventional absolutes obtained by extraction with hexane. The absolutes obtained at a temperature of −25° C. and at −20° C. for jasmine and −50° C. for lavender were preferred by the perfumers, who emphasised their delicacy and their power.

On the other hand, maceration at a temperature above −10° C. (e.g. −5° C., 10° C. or 25° C.) led to the production of highly coloured extracts having a flatter olfactive profile, less characteristic of the extracted flowers or even unpleasant.

For tonka beans, a good olfactive profile was obtained at −5° C.

Furthermore, the extractions carried out with too short maceration times gave unsatisfactory results from an olfactory point of view (Extract 41 of blackcurrant of 15 minutes, Extract 13 of mimosa of 2×10 minutes and extract 1 of jasmine of 10 minutes). These absolutes from incomplete extractions lack the characteristic olfactory notes of the plant and/or lack persistence overtime.

It is also noted that the yields are better for the process of extraction with cold ethanol than for the extraction with hexane. Fewer waxes are extracted in the process according to the invention, which facilitates the recovery of the absolute from the concrete.

### Example 3: Characterization of the Absolutes Obtained by the Process According to the Invention

GC FID Analysis

The extracts are characterized by analysis by gas chromatography coupled to a flame ionization detector (GC/FID). The analysis conditions are as follows:

Agilent HP-1 ms column (apolar, 100% dimethylpolysiloxane, 60 m×250 μm×0.25 μm), oven program: 60° C. for 10 minutes and then 2° C./minute up to 300° C. and plateau for 20 minutes.

The table below shows the distribution of the main volatile compounds present in the hexane absolute and the “ethanol” absolute according to the invention. For each compound, the percentage indicated corresponds to the area percentage of the peak corresponding to the chemical compound of interest relative to the total area of the peaks in the chromatogram obtained by GC/FID analysis.

Lavandula angustifolia, Saralia 22 and 29 Hybrids

TABLE 12

| | Low-temperature ethanol | |
|---|---|---|
| | absolute (two ethanolic | Hexane absolute |
| | macerations at −25° C., 30 min) | (comparative) |
| Terpenes, including: | 2.3% | 3.2% |
| BETA-CARYOPHYLLENE | 1.8% | 2.5% |
| Terpenoids, including: | 78.3% | 71.9% |
| LINALOOL | 43.7% | 39.6% |
| LINALYL ACETATE | 25.8% | 29.1% |
| CAMPHOR | 0.7% | 0.2% |
| BORNEOL | 2.8% | 0.6% |
| Phenolic compounds, including: | 2.1% | 18.1% |
| COUMARIN | 1.5% | 10.4% |

Jasminum grandiflorum

TABLE 13

| | Low-temperature ethanol | |
|---|---|---|
| | absolute (two ethanolic | Hexane absolute |
| | macerations at −25° C., 30 minutes) | (comparative) |
| Alcohols, including: | 1.47% | 0.19% |
| BENZYL ALCOHOL | 1.27% | 0.19% |
| Esters, including: | 13.00% | 28.12% |
| BENZYL ACETATE | 3.99% | 14.14% |
| BENZYL BENZOATE* | 6.67%* | 11.30% |
| CIS-TRANS-METHYL JASMONATE | 0.80% | 1.52% |
| Ketones/lactones, including: | 3.02% | 5.90% |
| CIS-JASMONE | 1.34% | 1.78% |
| CIS-JASMINE LACTONE | 0.90% | 1.52% |
| JASMINE KETOLACTONE | ND | 1.11% |
| Phenolic compounds, including: | 7.74% | 0.20% |
| EUGENOL | 0.91% | 0.20% |
| 4-HYDROXYPHENYLETHYL ALCOHOL | 0.69% | ND |
| HYDROXYTYROSOL | 6.14% | ND |
| Fatty acid esters | 2.50% | 2.54% |
| Total terpenes and terpenoids, including: | 36.32% | 42.75% |
| LINALOOL | 1.30% | 3.49% |
| TRANS-TRANS-ALPHA-FARNESENE | 1.80% | 2.28% |
| ISOPHYTOL | 6.85% | 8.58% |
| PHYTOL | 9.31% | 11.85% |
| SQUALENE | 3.99% | 2.46% |
| EPOXYSQUALENE | 6.67% | 7.06% |
| Other compounds, including: | 0.73% (+co-elution | ND |
| | with benzyl benzoate) | |
| ELENOLIDE ISOMERS INDOLE | 2.90% | 5.26% |

*co-eluted with an elenolide isomer;
ND: Not Detected

Mimosa

TABLE 14

| | | Hexane 25° C. | |
|---|---|---|---|
| | | 2 × 30 min | Ethanol −25° C. |
| Compounds | | (comparative) | 2 × 30 min |
| HEPTANAL | Aldehyde | 0.10% | — |
| 3,5-DIMETHYL-2-HEXANONE | Ketone | 0.11% | — |
| 3-HYDROXY-3-METHYLHEPTAN-5-ONE | Ketone | — | 0.70% |
| 3-HYDROXY-3-METHYLHEPTAN-5-ONE | Ketone | — | 0.37% |
| p-ANISIC ACID | Phenylpropanoid/benzenoid | — | 4.51% |
| PENTADECANE | Alkane | 0.14% | 0.19% |
| TRANS-4-TETRADECENOL | Oxygenated monoterpene | 0.15% | 0.29% |
| 8-Z-HEPTADECENE | Alkene | 8.43% | 16.92% |
| HEPTADECANE | Alkane | 1.35% | 2.15% |
| OCTADECANE | Alkane | 0.15% | 0.22% |
| NONADECENE | Alkene | 0.50% | 1.28% |
| NONADECANE | Alkane | 5.26% | 5.55% |
| EICOSANE | Alkane | 0.76% | 0.80% |
| HENEICOSANE | Alkane | 1.84% | 0.82% |
| TRICOSANE | Alkane | 1.53% | 0.39% |
| BETA-AMYRENONE | Oxygenated triterpene | 1.97% | 2.22% |
| GERMANICONE | Oxygenated triterpene | 10.56% | 7.89% |
| BETA-AMYRINE | Oxygenated triterpene | 1.05% | 0.46% |
| LUPENONE | Oxygenated triterpene | 35.58% | 27.77% |
| LUPEOL + ALPHA-AMYRINE | Oxygenated triterpene | 5.61% | 2.97% |
| MORETENONE | Oxygenated triterpene | 4.76% | 0.98% |
| MORETENOL | Oxygenated triterpene | 0.98% | * |
| TOTAL | Aliphatic aldehydes/ketones | 0.21% | 1.07% |
| | Alkanes/alkenes | 19.96% | 28.32% |
| | Phenylpropanoids/benzenoids | 0.00% | 4.51% |
| | Terpenes/terpenoids | 60.66% | 42.58% |

Rose

TABLE 15

| | | | Hexane 2 × 30 |
|---|---|---|---|
| | | Ethanol 1 × 30 | min, 25° C. |
| Compounds | | min, −25° C. | (Comparative) |
| BENZYL ALCOHOL | Phenylpropanoids/benzenoids | 0.90% | 0.72% |
| PHENETHYL ALCOHOL | Phenylpropanoids/benzenoids | 31.60% | 44.04% |
| NEROL | Oxygenated monoterpene | 1.65% | 3.09% |
| CITRONELLOL | Oxygenated monoterpene | 4.64% | 3.89% |
| GERANIOL | Oxygenated monoterpene | 4.65% | 7.83% |
| EUGENOL | Phenylpropanoids/benzenoids | 0.53% | 1.65% |
| METHYL EUGENOL | Phenylpropanoids/benzenoids | 0.29% | 0.36% |
| HEPTADECANE | Alkane | 1.35% | 0.27% |
| NONADECENE | Alkene | 2.64% | 0.61% |
| NONADECANE | Alkane | 4.97% | 0.87% |
| TOTAL | Alkanes/alkenes | 8.96% | 1.75% |
| | Phenylpropanoids/benzenoids | 33.32% | 46.77% |
| | Terpenes and terpenoids | 10.94% | 14.81% |

Immortelle

TABLE 16

| | | Hexane 3 × 45 |
|---|---|---|
| | Ethanol 1 × 30 | min, 30° C. |
| | min, −15° C. | (Comparative) |
| Phenolic compounds, including: | 12.97% | 3.81% |
| 1-(2,4,6-TRIHYDROXY-3-(3-METHYLBUT- | 12.25% | 2.89% |
| 2-EN-1-YL)PHENYL)ETHANONE | | |
| Total terpenes and terpenoids, including: | 12.68% | 32.64% |
| LIMONENE | 0.37% | 0.32% |
| LINALOOL | 0.09% | 0.51% |
| BORNEOL | 0.20% | 0.14% |
| ALPHA-TERPINEOL | 0.27% | 0.47% |
| NEROL | 0.25% | 1.07% |
| NERYL ACETATE | 0.28% | 2.02% |
| ALPHA-COPAENE | 0.36% | 1.13% |
| BETA-CARYOPHYLLENE | 1.26% | 2.31% |
| ALPHA-HUMULENE | 0.48% | 0.21% |
| ALPHA-CURCUMENE | 0.83% | 2.35% |
| GAMMA-CURCUMENE | 1.18% | 3.17% |
| BETA-SELINENE | 1.27% | 4.37% |
| SELINA-3,11-DIENE | 0.81% | 2.80% |
| TRANS-NEROLIDOL | 0.38% | 0.10% |
| GUAIOL | 0.13% | 0.27% |
| ROSIFOLIOL | 0.25% | 1.16% |
| GAMMA-EUDESMOL | 0.95% | 0.28% |
| BETA-EUDESMOL | 0.20% | 0.28% |
| ALPHA-EUDESMOL | 0.24% | 0.84% |
| BETA-BISABOLOL | 0.21% | 0.37% |
| SQUALENE | 0.38% | 0.67% |
| Fatty acid esters | 2.28% | 10.89% |

Clary Sage

TABLE 17

| | | | Hexane 1 × 30 |
|---|---|---|---|
| | | Ethanol 1 × 30 | min, 25° C. |
| Compounds | | min −25° C. | (comparative) |
| LINALOOL | Oxygenated monoterpene | 1.00% | 1.94% |
| ALPHA-TERPINEOL | Oxygenated monoterpene | 0.03% | 0.05% |
| LINALYL ACETATE | Terpenoid | 5.77% | 9.65% |
| BETA-CARYOPHYLLENE | Sesquiterpene | 0.10% | 0.16% |
| GERMACRENE D | Sesquiterpene | 0.24% | 0.39% |
| SCLAREOL OXIDE | Oxygenated triterpene | 0.31% | 0.15% |
| SCLAREOL | Oxygenated triterpene | 82.76% | 80.96% |
| TOTAL | Terpenes and terpenoids | 90.21% | 93.30% |

Pelargonium

TABLE 18

| | | | Hexane 1 × 30 |
|---|---|---|---|
| | | Ethanol 1 × 30 | min, 25° C. |
| Compounds | | min −15° C. | (comparative) |
| LINALOOL | Oxygenated monoterpene | 0.34% | 0.14% |
| CIS-ROSE OXIDE | Oxygenated monoterpene | 1.49% | 0.12% |
| TRANS-ROSE OXIDE | Oxygenated monoterpene | 0.66% | 0.04% |
| MENTHONE | Oxygenated monoterpene | 0.97% | 0.35% |
| ISOMENTHONE | Oxygenated monoterpene | 1.32% | 3.90% |
| CITRONELLOL | Oxygenated monoterpene | 21.93% | 24.92% |
| GERANIOL | Oxygenated monoterpene | 12.49% | 12.60% |
| GERANIAL | Oxygenated monoterpene | 0.31% | 1.43% |
| CITRONELLYL FORMATE | Terpenoid | 0.65% | 4.52% |
| GERANYL FORMATE | Terpenoid | 0.23% | 1.96% |
| GERMACRENE D | Sesquiterpene | 0.55% | 0.65% |
| 10-EPI-GAMMA-EUDESMOL | Oxygenated sesquiterpene | 3.89% | 1.54% |
| GERANYL TYGLATE | Terpenoid | 2.36% | 1.90% |
| TOTAL | Terpenes and terpenoids | 45.14% | 53.9% |

Tonka

TABLE 19

| | | | Nappar 6/isopropyl |
|---|---|---|---|
| | | Ethanol −5° C., | acetate 35° C., |
| Compounds | | 2 × 1 h | 2 × 1 h |
| 3,4-DIHYDROCOUMARIN | Phenylpropanoid/benzenoid | 0.69% | 1.70% |
| COUMARIN | Phenylpropanoid/benzenoid | 94.50% | 94.16% |
| COUMARIN (internal calibration) | | 76.30% | 72.00% |
| TOTAL | Phenylpropanoid/benzenoid | 95.19% | 95.86% |

Blackcurrant

TABLE 20

| | | | Dichloro- |
|---|---|---|---|
| | | Ethanol 2 × 30 | methane 2 × 30 |
| Compounds | | min, −15° C. | min, 25° C. |
| ALPHA-THUJENE | Monoterpene | 0.74% | 0.39% |
| SABINENE | Monoterpene | 0.95% | 1.44% |
| DELTA-3-CARENE | Monoterpene | 0.55% | 6.42% |
| GAMMA-TERPINENE | Monoterpene | 0.78% | 0.36% |
| ALPHA-PHELLANDREN-8 OL | Oxygenated monoterpene | 0.63% | 0.17% |
| TERPINOLENE | Monoterpene | 0.60% | 7.66% |
| TERPINENE-4-OL | Oxygenated monoterpene | 0.95% | 0.37% |
| PARA-CYMEN-8-OL | Phenylpropanoid/benzenoid | 3.03% | 1.61% |
| 4-HYDROXYPHENYLETHYL ALCOHOL | Phenylpropanoid/benzenoid | 1.96% | 0.47% |
| ALPHA-TERPINYL ACETATE | Terpenoid | 0.80% | 0.44% |
| BETA-CARYOPHYLLENE | Sesquiterpene | 9.73% | 13.54% |
| ALPHA-HUMULENE | Sesquiterpene | 4.74% | 5.16% |
| BETA-CUBEBENE | Sesquiterpene | 1.97% | 3.09% |
| BICYCLOGERMACRENE | Sesquiterpene | 0.96% | 2.40% |
| SPATHULENOL | Oxygenated sesquiterpene | 6.37% | 2.09% |
| BETA-CARYOPHYLLENE OXIDE | Oxygenated sesquiterpene | 6.14% | 1.62% |
| ALPHA-HUMULENE OXIDE | Oxygenated sesquiterpene | 2.71% | 0.67% |
| THUJOPSENE | Sesquiterpene | 7.81% | 6.53% |
| TOTAL | Terpenes/terpenoids | 33.32% | 48.64% |

### Example 4: Perfume Composition Incorporating an Absolute According to the Invention

TABLE 21

| Chemical name or commercial name | Parts/1000 |
|---|---|
| Essential oil of bergamot | 50 |
| Essential oil of lemon | 20 |
| Linalool | 30 |
| Hexyl acetate | 10 |
| Liffarome toco (IFF) | 5 |
| Geranyle acetate | 10 |
| Citronellyl acetate | 10 |
| Citronellol | 10 |
| Geraniol | 20 |
| Phenyl ethyl alcohol | 30 |
| Helional | 30 |
| Floriffol (ELINCS) Toco | 85 |
| Absolute of jasmine obtained by | 20 |
| maceration in ethanol at −25° C. | |
| according to the invention | |
| Benzyl acetate | 5 |
| Methyl dihydrojasmonate | 340 |
| Benzyl salicylate | 100 |
| Indole | 5 |
| Heliotropine (piperonal) | 5 |
| Iso gamma Super Toco (IFF) | 50 |
| Mysantol (IFF) | 15 |
| Cedramber (IFF) | 10 |
| Ambermor Ex (IFF) | 25 |
| Ethylene Brassylate (Astratone) | 70 |
| TOTAL | 1000 |

1-34. (canceled)

35. A process for preparing an absolute from a plant material for use as an ingredient in perfumery, cosmetics or in food industry, said process comprising a step of ethanolic extraction at a temperature below −10° C. of the plant material so as to extract the odorous molecules present in the plant material, the plant material being not obtained from genus Cannabis.

36. The process for preparing an absolute of claim 35, comprising:
a) a step of extracting the plant material in ethanol, at a temperature below −10° C. so as to extract the odorous molecules present in the plant material,
b) a step of separating the plant material and the ethanol so as to recover the ethanol containing odorous molecules,
c) a step of removing at least partially the ethanol, and
d) optionally, a step of removing waxes from the extract obtained in step c), whereby the absolute is obtained.

37. The process for preparing an absolute of claim 36, wherein step a) is performed by maceration of the plant material in ethanol at a temperature ranging from −15° C. to −50° C. for 15 to 120 minutes.

38. The process for preparing an absolute of claim 36, wherein step b) is performed by decantation, pressing, centrifugation and/or filtration of the plant material.

39. The process for preparing an absolute of claim 36, wherein steps a) and b) are repeated at least once.

40. The process for preparing an absolute of claim 36, wherein, in step c), the concentrated extract has a concentration of concrete of from 5% to 15% by volume in ethanol.

41. The process for preparing an absolute of claim 36, wherein, in step d), the waxes are removed by cold precipitation.

42. The process for preparing an absolute of claim 35, wherein the plant material is selected from fruit, nuts, buds, flowers, flowering tips, leaves, stems, branches, wood, bark, zests, roots, rhizomes, pips, seeds and pits and combinations and fractions thereof.

43. The process for preparing an absolute of claim 35, wherein the plant material is selected from buds, flowers, flowering tips, leaves, stems and branches.

44. The process for preparing an absolute according to claim 35, wherein the plant material is a fresh material.

45. The process for preparing an absolute of claim 35, wherein the plant material is obtained from a species selected from the genera Lavandula, Jasminum, Rosa, Narcissus, Acacia, Ribes, Iris, Citrus, Polianthes, Pogostemon, Ocimum, Pistacia, Myristica, Tagetes, Ilex, Osmanthus, Mentha, Gardenia, Convallaria, Cinnamonum, Pelargonium, Viola, Gentiana, Triticum, Cananga, Lilium, Hyacinthus, Dianthus, Vanilla, Quercus, Cupressus, Helichrysum, Salvia, Spartium, Cistus, Evernia and Lolium.

46. The process for preparing an absolute of claim 35, wherein the plant material is obtained from a plant species selected from Helichrysum, Salvia, Lavandula, Jasminum, Rosa, Acacia, Ribes, and Pelargonium.

47. The process for preparing an absolute of claim 35, wherein the resulting absolute is a fragrance for cosmetic or perfume composition, or a flavour for food product.

48. The process for preparing an absolute of claim 35, wherein the plant material is obtained from a species of jasmine, a species of lavender or a species of rose.

49. The process for preparing an absolute of claim 35, the odorous molecules that are extracted comprise terpenic compounds, terpenoid compounds, benzenoid derivatives, phenylpropanoid derivatives, phenolic compounds and combinations thereof.

50. An absolute obtainable by the process as defined in claim 35.

51. The absolute of claim 50, which is an absolute of jasmine flowers comprising at least 0.3% of elenolide isomers and at least 3.0% of hydroxytyrosol.

52. The absolute of claim 50 characterized in that it comprises at least 30% of terpenic compounds and is selected from an absolute of jasmine, of lavender, of mimosa, of clary sage, of pelargonium and of blackcurrant.

53. The absolute of claim 50, which is an absolute from rose flowers or an absolute from immortelle flowers, leaves and stems.

54. The absolute of claim 50 which is selected from the group consisting of a cosmetic ingredient, a fragrance, a cosmetic active agent and a flavour ingredient for food products.
